(19) United States
(12) Patent Application Publication
(10) Pub. No.: US 2007/0093905 A1
(43) Pub. Date: Apr. 26, 2007

(54) Degenerative disc regeneration techniques

(76) Inventors: Michael J. O'Neil, W. Barnstable, MA (US); Mohammed Attawia, Canton, MA (US); Thomas M. DiMauro, Southboro, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/255,375

(22) Filed: Oct. 21, 2005

(51) Int. Cl. A61F 2/44

(52) U.S. Cl. 623/17.16; 623/919

(57) ABSTRACT

In the repair/regenerate of the intervertebral disc, all or a portion of the nucleus pulposus or annulus fibrosus is excised and treated for reinsertion into the disc or adjacent or alternate level disc. Alternatively certain bioactive agents may be injected into the degenerative disc without excising disc material.

### BACKGROUND OF THE INVENTION

1. Field of the Invention

This invention is concerned with methods for repair and regeneration of intervertebral discs afflicted with degenerative disc disease.

2. Related Art

The natural intervertebral disc contains a jelly-like nucleus pulposus surrounded by a fibrous annulus fibrosus. Under an axial load, the nucleus pulposus compresses and radially transfers that load to the annulus fibrosus. The laminated nature of the annulus fibrosus provides it with a high tensile strength and so allows it to expand radially in response to this transferred load.

In a healthy intervertebral disc, cells within the nucleus pulposus produce an extracellular matrix (ECM) containing a high percentage of proteoglycans. These proteoglycans contain sulfated functional groups that retain water, thereby providing the nucleus pulposus with its cushioning qualities. These nucleus pulposus cells may also secrete small amounts of cytokines such as IL-lβ as well as matrix metalloproteinases (MMPs). These cytokines and MMPs help regulate the metabolism of the nucleus pulposus cells.

In some instances of degenerative disc disease (DDD), (the gradual degeneration of the intervertebral disc) is caused by mechanical instabilities in other portions of the spine. In these instances, increased loads and pressures on the nucleus pulposus cause the cells to emit larger than normal amounts of the above-mentioned cytokines. In other instances of DDD, genetic factors, such as programmed cell death, or apoptosis can also cause the cells within the nucleus pulposus to emit toxic amounts of these cytokines and MMPs. In some instances, the pumping action of the disc may malfunction (due to, for example, a decrease in the proteoglycan concentration within the nucleus pulposus), thereby retarding the flow of nutrients into the disc as well as the flow of waste products out of the disc. This reduced capacity to eliminate waste may result in the accumulation of high levels of toxins.

As DDD progresses, the toxic levels of the cytokines present in the nucleus pulposus begin to degrade the extracellular matrix. In particular, the MMPs (under mediation by the cytokines) begin cleaving the water-retaining portions of the proteoglycans, thereby reducing their water-retaining capabilities. This degradation leads to a less flexible nucleus pulposus, and so changes the load pattern within the disc, thereby possibly causing delamination of the annulus fibrosus. These changes cause more mechanical instability, thereby causing the cells to emit even more cytokines, typically thereby upregulating MMPs. As this destructive cascade continues and DDD further progresses, the disc begins to bulge (“a herniated disc”), and then ultimately ruptures, ejecting the nucleus pulposus from the disc and causing it to contact a local nerve root and produce sciatic pain.

US20050074477, describes culturing disc cells and implanting the cultured cells into the damaged disc.

In U.S. Pat. No. 6,685,695 Ferree discloses the use of porous stents as a method to provide nutrients to the disc by forming a passageway through vertebral end plate and providing one or more substances beneficial to the disc.

US20040083001 disclosed the creation of an engineered biological material comprising, one or more tissue of the intervertebral disc.

U.S. Pat. No. 6,340,369, Ferree discloses the use of harvested live, disc cells combined with an ECM and transplanted into the disc.

However, none of the prior art appears to describe the “salvaging” of herniated nucleus pulposus or annulus fibrosus for treatment and immediate intra-operative reinsertion or intraopertive processing and reinsertion in to the operative level or alternate level degenerated disc as hereinafter disclosed. Also disclosed are the benefits of injection of amino acids such as glycine as a promoter for anti-inflammatory production either with the disc material to be reinserted or by separate injection without the disc material.

### BRIEF DESCRIPTION OF THE DRAWINGS

FIG. 1 depicts a herniated disc.

FIGS. 2-5 depict a disc under repair with various places where the treated excised tissue may be reinserted into the disc.

### SUMMARY OF THE INVENTION

This invention is generally related to allograph annulus fibrosus and/or nucleus pulposus tissue which may be removed from a degenerative disc during discectomy, herniactomy, or nucleutomy and treated by one of several means to isolate cells. The isolated cells may be further treated by mixing with bioactive agents such as growth factors and/or anti-inflammatory agents then inserted or re-injected into the disc space. This procedure could be followed by annular repair plug in order to avoid disc tissue herniation.

Thus one embodiment of the invention is directed to a method of treating a degenerative intervertebral disc having a nucleus pulposus and annulus fibrosus, comprising the steps of:

a) excising all or a portion of the degenerative nucleus pulposus or annulus fibrosus;

b) treating the excised nucleus pulposus or annulus fibrosus; and

c) reintroducing the treated nucleus pulposus or annulus fibrosus into the disc.

Another embodiment of the invention relates to a method of treating a degenerative intervertebral disc having a nucleus pulposus and a damaged annulus fibrosus, comprising the steps of:

a) excising all or a portion of the nucleus pulposus or annulus fibrosus;

b) treating the excised nucleus pulposus or annulus fibrosus;

c) plugging the site of the damaged annulus fibrosus with a resealable plug; and

d) reintroducing the nucleus pulposus or annulus fibrosus into the nucleus of the disc through the plug.

Alternate embodiments include: (i) reintroducing the treated nucleus pulposus or annulus fibrosus in to the disc and then sealing the annulus fibrosus with a plug and (ii) reintroducing the treated nucleus pulposus or annulus fibrosus into the disc through an adjacent vertebral body.

Yet other embodiments relate to the use of glycine or any glycine-like amino acids in treating degenerative disc disease.

Prior art does disclose the use of live disc cells, but no art discloses separation of excised allograph tissue and mixing with bioactive agents such as growth factors, amino acids, and/or anti-inflammatory agents.

### DETAILED DESCRIPTION OF PREFERRED EMBODIMENTS OF THE INVENTION

An intervertebral disc herniation or bulge may be removed via currently available mechanical means (ronguers) or via aspiration through either adjacent vertebral body.

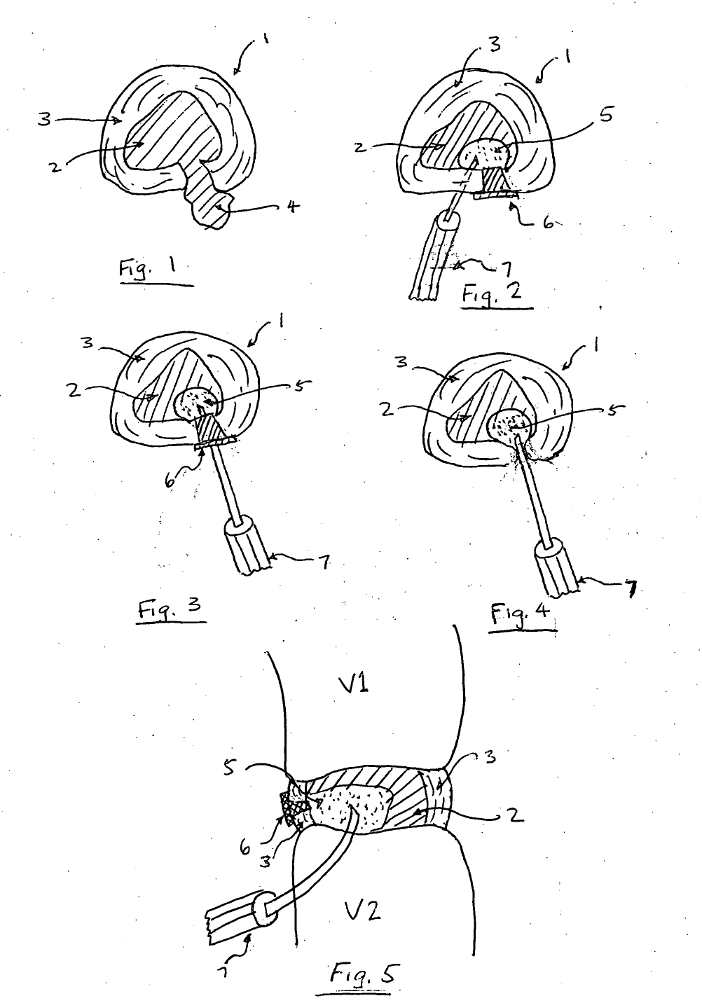

FIG. 1 depicts a cross-sectional view of herniated disc 1 comprising nucleus pulposus 2, annulus fibrosus or annular ring 3 and a nucleus pulposus 4 extending through a defect in the annulus fibrosus 3 outside of disc 1.

In the method of this invention, all or a portion of nucleus pulposus 2 or a portion of the annulus fibrosus is removed creating void for reintroduction of the treated tissue.

FIGS. 2 to 5 depict disc 1 with a portion of nucleus pulposus 2 and annulus fibrosus 3 removed and void 5 into which the treated tissue is reintroduced by device 7 such as a cannula, syringe or any other suitable tissue delivery device. Thus, FIGS. 2 to 5 support the some of the various methods that repair of intervertebral disc 1 may be accomplished.

Specifically, FIG. 2 depicts reintroduction of treated tissue into void 5 by use of device 7 through annulus fibrosus 3 with plug 6 sealing annulus fibrosus 3.

FIG. 3 depicts reintroduction of treated tissue into void 5 through use of device 7 through plug 6.

FIG. 4 shows reintroduction of treated tissue into void 5 by use of device 7 through an opening in annulus fibrosus 3.

FIG. 5 depicts reintroduction of treated tissue into void 5 through use of device 7 through adjacent vertebral body V2.

The removed disc cells or annulus are treated via mechanical means and/or enzymatic degradation.

Examples of suitable types if mechanical separation techniques that the excised disc tissue can be processed and isolated into viable disc cells include mincing, chopping, slicing, milling, morselizing, pulverizing, shearing, grinding, trimming, stripping, skinning as non-limiting examples. Disc cell isolation can be further facilitated with the use of other known separation techniques including filters, centrifuges, separation columns, or affinity columns, for example.

Examples of suitable enzymes useful to encourage disc cell isolation include, but are not limited to, collagenase, chondroitinase, trypsin, elastase, hyaluronidase, peptidase, thermolysin, matrix metalloproteinase, EDTA, gelatinase and protease. Preferred enzymes are collagenase, trypsine, and EDTA.

The isolated disc cells may then be mixed with bioactive agents such as growth factors, amino acids, and/or anti-inflammatory agents. The resulting mixture is inserted or injected into the disc either through or into the annular wall defect or through an adjacent vertebral body.

An optional plug is inserted into the disc defect to prevent release of the disc regeneration material.

The disc regeneration material encourages regeneration of the disc through the bioactive agent(s)' (such as growth factor or anti-inflammatory agents) interactions with the cellular materials. Anti-inflammatory agents reduce biologic responses, retard disc degeneration and further inducing tissue regeneration.

The reinserted nucleus or annular tissue can also optionally be combined with a variety of other materials, including carriers (which may in and of themselves possess bioactivity or have a propensity to cause bioactivity when combined with other agents and hence may also serve as a bioactive agent), such as a gel-like carrier or an adhesive. By way of non-limiting example, the gel-like carrier can be a biological or synthetic hydrogel, hyaluronic acid, buffered saline, fibrin glue, fibrin clot, collagen gel, collagen-based adhesive, alginate gel, crosslinked alginate, chitosan, synthetic acrylate-based gels, platelet rich plasma (PRP), platelet poor plasma (PPP), PRP clot, PPP clot, blood, blood clot, blood component, blood component clot, Matrigel, agarose, chitin, chitosan, polysaccharides, poly(oxyalkylene), a copolymer of poly(ethylene oxide)-poly(propylene oxide), poly(vinyl alcohol), laminin, elastin, proteoglycans, solubilized basement membrane, or combinations thereof. Suitable adhesives include, but are not limited to, hyaluronic acid, fibrin glue, fibrin clot, collagen gel, collagen-based adhesive, alginate gel, crosslinked alginate, gelatin-resorcin-formalin-based adhesive, mussel-based adhesive, dihydroxyphenylalanine (DOPA)-based adhesive, chitosan, transglutaminase, poly(amino acid)-based adhesive, cellulose-based adhesive, polysaccharide-based adhesive, synthetic acrylate-based adhesives, platelet rich plasma (PRP), platelet poor plasma (PPP), PRP clot, PPP clot, blood, blood clot, blood component, blood component clot, polyethylene glycol-based adhesive, Matrigel, Monostearoyl Glycerol co-Succinate (MGSA), Monostearoyl Glycerol co-Succinate/polyethylene glycol (MGSA/PEG) copolymers, laminin, elastin, proteoglycans, and combinations thereof.

Additionally, bioactive agents may also be combined with the nucleus or annular material to be reinserted.

“Bioactive agents,” as used herein, can include one or more of the following: chemotactic agents; therapeutic agents (e.g., antibiotics, steroidal and non-steroidal analgesics and anti-inflammatories (including certain amino acids such as glycine), anti-rejection agents such as immunosuppressants and anti-cancer drugs); various proteins (e.g., short term peptides, bone morphogenic proteins, collagen, hyaluronic acid, glycoproteins, and lipoprotein); cell attachment mediators; biologically active ligands; integrin binding sequence; ligands; various growth and/or differentiation agents and fragments thereof (e.g., epidermal growth factor (EGF), hepatocyte growth factor (HGF), vascular endothelial growth factors (VEGF), fibroblast growth factors (e.g., bFGF), platelet derived growth factors (PDGF), insulin derived growth factor (e.g., IGF-1, IGF-II) and transforming growth factors (e.g., TGF-β I-III), parathyroid hormone, parathyroid hormone related peptide, bone morphogenic proteins (e.g., BMP-2, BMP-4; BMP-6; BMP-7; BMP-12; BMP-13; BMP-14), sonic hedgehog, growth differentiation factors (e.g., GDF5, GDF6, GDF8), recombinant human growth factors (e.g., MP52, and MP-52 variant rhGDF-5), cartilage-derived morphogenic proteins (CDMP-1; CDMP-2, CDMP-3)); small molecules that affect the upregulation of specific growth factors; tenascin-C; hyaluronic acid; chondroitin sulfate; fibronectin; decorin; thromboelastin; thrombin-derived peptides; heparin-binding domains; heparin; heparan sulfate; DNA fragments and DNA plasmids. Suitable effectors likewise include the agonists and antagonists of the agents described above. The growth factor can also include combinations of the growth factors described above. In addition, the growth factor can be autologous growth factor that is supplied by platelets in the blood. In this case, the growth factor from platelets will be an undefined cocktail of various growth factors. If other such substances have therapeutic value in the orthopaedic field, it is anticipated that at least some of these substances will have use in the present invention, and such substances should be included in the meaning of “bioactive agent” and “bioactive agents” unless expressly limited otherwise. Preferred examples of bioactive agents include culture media, bone morphogenic proteins, growth factors, growth differentiation factors, recombinant human growth factors, cartilage-derived morphogenic proteins, hydrogels, polymers, antibiotics, anti-inflammatory medications, immunosuppressive mediations, autologous, allogenic or xenologous cells such as stem cells, chondrocytes, fibroblast and proteins such as collagen and hyaluronic acid. Bioactive agents can be autologus, allogenic, xenogenic or recombinant.

The bioactive agents can take the form of immediate release (injection) or delayed release using microspheres, nanospheres or other matrices such as hydrogels for controlled release delivery to encourage disc tissue incorporation and regeneration.

In addition or as an alternative to the bioactive agents, healthy, viable cells may be added to the excised disc material to be reinserted. Examples of such cells include harvested cells selected from the group consisting of healthy nucleus pulposus or annulus fibrosus cells, precursors of nucleus pulposus or annulus fibrosus cells, or cells capable of differentiating into nucleus pulposus or annulus fibrosus cells.

It is envisioned that any suitable annular closure technique may be used before or after reinsertion of disc tissue. The annular closure technique can be applied before or after disc tissue implantation.

Examples of suitable closure techniques may include the use of the following alone or in combination, sutures (resorbable or non-resorbable strips/cords/draw strings/wires/cords), adhesives (fibrin, cyanoacrylates, polyanhydrides, glutaraldehydes, PRP, etc.), in-situ fabricated plugs (single sheet wound or two piece snapped together), pre-fabricated plugs (like a tire plug), expandable plugs (stent like), for example.

Delivery of the material to be reinserted into the nucleus pulposus or annulus fibrosus of the disc may be through the ruptured area of the annulus, by a separate passageway way through or into the annulus, or through a plug or other closure device used to repair the ruptured annulus. This invention also contemplates that all or a portion of the treated tissue can be injected into one or more adjacent or alternate spinal disc level.

It has been reported by Spittler, FASEB J., 13, 563-571 (1999) that monocytes cultured in glycine for 40 hours produce about 5 ng/ml of IL-10 (and only about 0.2 ng/ml TNF-a) when subsequently exposed to LPS. Thus, it is contemplated by the inventors that glycine may have beneficial effects on a degenerating disc and may be combined with the nucleus pulposis or annulus fibrosus material to be reinserted or be injected into the degenerating disc.

Glycine

U.S. Pat. No. 6,812,211 (Slivka) discloses injecting glycine (as an inactivating agent) into the intervertebral disc in an amount sufficient to substantially inactivate a crosslinking agent. Slivka does not disclose a) injecting a formulation comprising glycine and monocytes into an intervertebral disc in an amount sufficient to produce a therapeutic amount of IL-10.

UVB Light

In some embodiments, the excised nucleus pulpous or annulus fibrosus is treated with an effective amount of UVB light. The UVB light will therapeutically act upon any white blood cells (such as macrophages or T cells) that have infiltrated the disc tissue. This treatment will have the effect of shifting the immune response associated with the macrophages or T cells from a pro-inflammatory Th1 response to an anti-inflammatory Th2 response. In some embodiments, the UV light source is situated to irradiate adjacent tissue with between about 0.02 J/cm² and 20 J/cm² energy. Preferably, the light source has a spectral maximum in the range of the UVB component of the solar spectrum, which is between 280 nm and 320 nm. In some embodiments, the light source has a spectral maximum of about 311 nm-312 nm.

Some non-limiting examples follow.

### EXAMPLE 1

A mixture of glycine, concentrated monocytes and fibrin glue is injected into a degenerating disc (or outside the disc for sciatica). The fibrin glue isolates the glycine and monocytes from the disc material, thereby allowing in vivo culturing for a day or so. When the fibrin glue disappears after about 40 hours, the cultured monocytes will encounter disc-related antigen and produce IL-10. The IL-10 will then act as an anti-inflammatory in the disc.

In addition, glycine is an inhibitor of glutamate and it has been reported that glutamate may leak out of a degenerating disc and cause pain. In our process, the glycine that leaves the fibrin glue will inhibit the pain-causing actions of glutamate.

### EXAMPLE 2

A portion of the degenerating disc is excised and the antigenic cells removed. Glycine is combined with the antigenic cells and the combination is re-injected into the disc.

It should be noted that sustained-release forms of glycine may be used. For example, glycine will have the effect of significantly reducing TNF-a production within the disc for controlled periods (or from the extruded nucleus pulposus in sciatica) while only slightly lowering IL-10 production. Glycine can be made a controlled release substance by combining with such items as by adding to typical carriers including microspheres, foams, gels, and other much materials known in the art that permit sustained release kinetics.

It should be understood that the foregoing disclosure and description of the present invention are illustrative and explanatory thereof and various changes in the size, shape and materials as well as in the description of the preferred embodiment may be made without departing from the spirit of the invention.

What is claimed is:

1. A method of treating a degenerative intervertebral disc having a nucleus pulposus and annulus fibrosus, comprising the steps of:
a) excising all or a portion of the degenerative nucleus pulposus or annulus fibrosus;
b) treating the excised nucleus pulposus or annulus fibrosus; and
c) reintroducing the treated nucleus pulposus or annulus fibrosus into the disc.

2. The method of claim 1, wherein the step of reintroducing the nucleus pulposus or annulus fibrosus is at the site of herniation.

3. The method of claim 1, wherein the step of treating is selected from the group consisting of mechanical techniques and degradation by treatment of enzymes.

4. The method of claim 3, wherein the step of treating further comprises adding one or more bioactive agents.

5. The method of claim 4, wherein the bioactive agents are selected from the group consisting culture media, bone morphogenic proteins, growth factors, growth differentiation factors, recombinant human growth factors, cartilage-derived morphogenic proteins, hydrogels, polymers, antibiotics, anti-inflammatory medications, immunosuppressive mediations, autologous, allogenic or xenologous stem cells.

6. The method of claim 5, wherein the bone morphogenic proteins are selected from the group consisting of BMP-2, BMP-4, BMP-6, BMP-7, BMP-12, BMP-13, and BMP-14.

7. The method of claim 6, wherein the BMP is BMP-2.

8. The method of claim 6, wherein the BMP is BMP-7.

9. The method of claim 5, wherein the growth differentiation factor is selected from the group consisting of GDF5, GDF6 and GDF8.

10. The method of claim 5, wherein the recombinant human growth factor is rhGDF-5.

11. The method of claim 5, wherein the cartilage-derived morphogenic proteins are selected from the group consisting of CDMP-1, CDMP-2 and CDMP-3.

12. The method of claim 11, wherein the cartilage-derived morphogenic proteins is CDMP-1.

13. The method of claim 1, wherein the step of treating further comprises the addition of harvested cells selected from the group consisting of healthy nucleus pulposus or annulus fibrosus cells, precursors of nucleus pulposus or annulus fibrosus cells, or cells capable of differentiating into nucleus pulposus or annulus fibrosus cells.

14. The method of claim 1, further comprising the step of:
d) closing the annulus fibrosus with a plug.

15. The method of claim 1 wherein the excised nucleus pulposus or annulus fibrosus comprises infiltrated white blood cells, and wherein the treating step includes irradiating the white blood cells with an effective amount of UVB light.

16. A method of treating a degenerative intervertebral disc having a nucleus pulposus and a damaged annulus fibrosus, comprising the steps of:
a) excising all or a portion of the nucleus pulposus or annulus fibrosus;
b) treating the excised nucleus pulposus or annulus fibrosus;
c) plugging the site of the damaged annulus fibrosus with a resealable plug; and
d) reintroducing the nucleus pulposus into the nucleus of the disc through the plug.

17. The method of claim 1, wherein step c) is through an adjacent vertebral body.

18. A method of treating a degenerative intervertebral disc having a nucleus pulposus and annulus fibrosus, comprising the steps of:
a) excising all or a portion of the degenerative nucleus pulposus or annulus fibrosus;
b) treating the excised nucleus pulposus or annulus fibrosus; and
c) reintroducing all or a portion of the treated nucleus pulposus or annulus fibrosus into a second disc at an adjacent or alternate disc level.

19. A method of treating a degenerative intervertebral disc, comprising the step of inserting a composition comprising glycine, concentrated monocytes and fibrin glue into the disc.

20. A method for treating a degenerating intervertebral disc, comprising the steps of:
a) excising a portion of degenerative disc containing antigenic cells;
b) isolating the antigenic cells;
c) combining the antigenic cells with glycine; and
d) injecting the combined antigenic cells and glycine into the disc.
